(19) United States
(12) Patent Application Publication
(10) Pub. No.: US 2018/0122502 A1
(43) Pub. Date: May 3, 2018

(54) SYSTEM AND METHOD FOR CONTROLLING THE ADMINISTRATION OF A DRUG TO A PATIENT

(71) Applicant: Fresenius Vial SAS, Brézins (FR)

(72) Inventors: Alaster Jones, Saint Sauveur (FR); Olivier Dervyn, Tullins Isere/R.A. (FR)

(21) Appl. No.: 15/568,920

(22) PCT Filed: Apr. 13, 2016
(86) PCT No.: PCT/EP2016/058118

§ 371 Date: Oct. 24, 2017

(30) Foreign Application Priority Data
Apr. 27, 2015 (EP) EP15305641.1

(51) Int. Cl. G16H 20/17

(52) U.S. Cl. CPC G16H 20/17

(57) ABSTRACT

A system for controlling the administration of a drug to a patient comprises at least one medical device (11, 12, 12′) for administering a drug to a patient, a distribution server (14), and a drug library database (15) comprising drug library data (150), the medical device (11, 12, 12′) being programmable, according to drug library data (150) of the drug library database (15), by entering configuration data (151) into the medical device (11, 12, 12′) for performing an administration operation for administering a drug to a patient. Herein, the system is operative to record said configuration data (151) and to report said configuration data (151) to the distribution server (14), wherein the distribution server (14) is constituted to pass said configuration data (151) to an expert system (16) comprising a knowledge database (160) for storing said configuration data, the expert system (16) being constituted to evaluate the knowledge database (160) for modifying said drug library database (15) according to said evaluation. In this way a system for administering a drug to a patient is provided which allows to adapt a drug library according to the actual use of medical devices, in particular according to configuration data entered into medical devices for programming the medical devices.

The invention relates to a system and a method for controlling administration of a drug to a patient.

A system for administering a drug to a patient comprises at least one medical device for administering a drug to a patient, a distribution server and a drug library database comprising drug library data. The drug library database can be accessed by the distribution server such that drug library data can for example be transferred from the drug library database via the distribution server to the at least one medical device, and the medical device can be programmed, according to drug library data of the drug library database, by entering configuration data for performing an administration operation for administering a drug to a patient.

Generally, the drug library database may reside on a (separate) database server. It however is also conceivable that the drug library database is installed on and hence is part of the distribution server.

Typically, medical devices for administering a drug to a patient, such as infusion pumps, are installed at various locations in a healthcare environment, for example in a hospital. Such medical devices may for example be located in different rooms of wards of a hospital or in operating rooms. Nowadays, such medical devices are connected to a local network for communicating via wired or wireless means with a hospital management system hosted on a server located in the healthcare environment. For example, a group of infusion pumps may be installed on a rack serving as a communication link to the local network such that via the rack the infusion pumps are connected to the local network and are operative to communicate with a hospital management system on a server within the healthcare environment, for example within a hospital, via the local network, for example a local area network (LAN) or a wireless local area network (WLAN), or a single pump may have a wireless or wired communication connection to the local network and a hospital information system.

To control the operation of medical devices of this kind for administering drugs to a patient, so called drug libraries are used, a drug library comprising drug library data characterizing a drug, its ingredients, rules for compatibility and rules for administration or the like. A version of a drug library may locally be installed on the medical device, or—alternatively—a drug library may be referenced on a server hosting the drug library database, in that the medical device queries the drug library database in response to a selected medication being programmed by a user. A drug library may for example comprise a list of drugs in which each drug is associated with parameters defining for example boundary values for administration by means of an infusion device. Such boundary values may for example relate to a minimum and maximum dosage for administering a particular drug, a minimum and maximum rate for administering a drug, a minimum and maximum time of administration and the like. In addition, the boundary values may be dependent on the age, weight and gender of a patient and, hence, may be patient-specific.

By using drug libraries the operation of a medical device such as an infusion pump for administering a particular drug to a patient is controlled in that the medical device may be operated by a nurse only within the boundaries posed by the drug library. For this, for administering a drug to a patient, the nurse identifies the drug to the medical device, upon which the medical device automatically loads the respective rules and boundary values from a drug library installed on the medical device or queried on a server.

The drug libraries may for example be locally installed within a healthcare environment, for example within a hospital (or group of hospitals). The drug libraries are for example installed as software on a so-called distribution server within a hospital, or accessed through the distribution server, via which the drug library may then be distributed to medical devices located in different wards of a hospital in order to be installed on the medical devices.

Typically, the creation of a drug library is cumbersome. For creating a drug library, a large number of drug boundary values, such as a minimum and maximum infusion rate, a minimum and maximum dosage, a minimum and maximum duration of administration and the like must be entered, wherein for different medical uses of a drug the boundary values may vary. For example, if a particular drug may be used on cardiac patients as well as on patients having influenza, the boundary values for administering the drug may differ significantly such that for different medical uses of a drug different boundary values must be entered. In addition, the boundary values may depend on the particular constitution of a patient, for example the age, the gender or the weight of the patient. Because for this reason a huge amount of information is to be entered into a drug library, the creation of a drug library is time-consuming and possibly prone to errors.

In addition, the information entered into a drug library may differ from information a user has gained from his own experience. For example, a user may want to use a drug on a patient by setting an infusion rate or dosage which may fall out of the limits set by the drug library. This, according to rules set by the drug library, may however be prohibited, such that the user is not able to set and use the desired settings. The drug library may hence confine a user to settings which are too limited or may force the medical device to work outside of the boundaries considered acceptable, for example, by a physician or pharmacist.

In another scenario, the drug library may define a range of settings, for example, a range between a minimum and maximum infusion rate or the like, which is too large. Within actual use of a drug, a user may actually use an infusion rate only within a much smaller range forming a sub-range of the range defined by the drug library.

All this may depend on the actual use of infusion devices in particular environments. For example, in one ward of a hospital other settings may be commonly used than in another ward of the same hospital or in a different hospital.

There hence is a desire to be able to adapt a drug library according to the actual use of medical devices in a particular healthcare environment.

WO 2012/054657 A2 discloses a method for performing mobile medical information management. A web-based patient portal is accessed and medical history records of a patient are downloaded therefrom, based upon a user profile including personal information that uniquely identifies the patient. The download data can be output to a patient or a physician treating the patient.

Within a method known from WO 2007/126948 A2 a medication management unit associated with a medical device performs a medication order for administering a drug to a patient. The medication management unit identifies the medical device such as an infusion pump by means of its network IP address and monitors the general physical location of the medical device. A medication order is transmitted to the medication management unit, which then performs the medication order.

EP 2 410 448 A2 describes an infusion pump system in which questions regarding the effects of administration of a drug are provided and answers to those questions are transmitted to an attending staff. The answers are recorded and the pump is controlled in that the dose of the drug is adjusted in accordance with the answers. For a safe remote programming of a pump infusion system, a predetermined program is used, and a protocol to be programmed is compared with a protocol coming from the pump.

It is an object of the instant invention to provide a system and a method for controlling the administering of a drug to a patient which allow to adapt a drug library according to the actual use of medical devices, in particular according to configuration data entered into medical devices for programming the medical devices.

This object is achieved by means of a system according to the features of claim 1.

Accordingly, the system is operative to record configuration data and to report said configuration data to the distribution server, wherein the distribution server is constituted to pass said configuration data to an expert system comprising a knowledge database for storing said configuration data, the expert system being constituted to evaluate the knowledge database for modifying said drug library database according to said evaluation.

Generally, the distribution server, drug library database and the expert system may reside on different server systems. In this case the distribution server serves as link for distributing drug library data to the medical devices and to relay back configuration data to the expert system. Alternatively, the drug library database and/or the expert system may be installed on the distribution server and hence may reside on the distribution server.

The medical device may in particular be an infusion device for infusing a medical liquid to a patient, in particular, an infusion pump such as a volumetric infusion pump or a syringe infusion pump.

The medical device also may be a rack on which a multiplicity of infusion devices can be arranged. Such rack may be constituted to organize the infusion devices by providing a mechanical connection for the infusion devices and by, in addition, providing a communication link via which the infusion devices can communicate with a communication network, for example within a healthcare environment, in particular a hospital communication network of a hospital.

The medical device may also be a monitor station for monitoring and/or controlling an infusion operation attached to the rack or connected to the distribution server via the network.

By means of the knowledge database on or accessed by the distribution server it becomes possible to automatically learn details about actual use of drugs, for example within a particular healthcare environment. By means of the expert system comprising the knowledge database it becomes possible to reduce errors and to improve an existing drug library database by modifying settings and rules stored therein. The modifications herein may be carried out by hospital, by ward or by healthcare area, by nurse, by patient profile, by drug name or the like.

The workflow which may be provided by the system may be as follows:

At an initial time, an initial drug library may be installed on a particular medical device, for example an infusion device, from the distribution server. The distribution server herein may be constituted to push the drug library from its drug library database to the medical device such that the installation at the medical device may take place automatically via the distribution server. The installation on the medical device, however, may also take place in another way, for example via an installation that is initiated from the medical device, or by using an external device which receives the drug library from the distribution server and then transmits the drug library to the medical device.

In a specific embodiment, a medical device may for example pull drug library data from the drug library database by queering a unique identifier of an available version of a drug library in the drug library database and by comparing the received unique identifier against a unique identifier of a local copy currently installed on the medical device. If the unique identifiers do not match, an updated version of the drug library may be installed on the medical device.

In another embodiment, a drug library may actually not be installed on the medical device directly, but the medical device may refer to drug library data contained and located in the drug library database. When a user wishes to use a medical device and for this programs the medical device, the medical device may query the drug library database, and the programming may take place within the boundaries and settings posed by a referenced drug library in the drug library database.

Each time the medical device, then, is used by a user for administering a drug to a patient, information entered into the medical device for programming an actual administration operation is recorded and reported to the knowledge database via the distribution server. The recording takes place at the medical device, and the information is relayed to the expert system comprising the knowledge database either instantaneously or periodically. The configuration data entered by a user to program the medical device for an actual administration operation for example may relate to a particular patient and its constitution, in particular a drug, the location of the medical device in a healthcare environment, a ward name, a nurse, a medical device ID and type or the like. The configuration data may furthermore relate to the actual settings for performing the administration operation, for example an infusion rate, a dosage and a time duration of infusion. Such information is recorded and is reported for example to the distribution server, which relays the information to the expert system.

It is to be noted that the programming of a medical device may take place by entering configuration data directly into the medical device which for this purpose may for example comprise a suitable input device such as a touch screen. The programming of a medical device may however also take place via an external system which for example may belong to a pharmacy or may be a so called auto-programming system. From such external systems configuration data may be transmitted to the medical device via the distribution server and in this way may be entered into the medical device.

After repeated uses of one or several medical devices within a healthcare environment, for example within a ward, a care area or an entire hospital, all information that has been entered into the knowledge database may mathematically, for example statistically, be analysed. According to this analysis, it may be found that particular settings of a drug library in the drug library database of the distribution server may actually be inaccurate. According to the analysis, hence, the drug library database may be modified in order to improve its accuracy. The modification could be proposed for confirmation by a user or could be implemented automatically. In the former case, the user may be given the option to challenge the proposition and to provide an alternative.

For example, it may be found that a particular drug is commonly used for example with an infusion rate within a range much smaller than set by the boundary values stored in the drug library database. Accordingly, the boundary values for this particular drug may be modified within the drug library database according to the actual range within which the actually used infusion rates lie.

This may take place dependent for example on patient demographics. For example, it may be found that for a new born the infusion rate as it actually is used within the healthcare environment on the medical device lies within a particular range.

Upon the analysis, it may also be found that users often desire to use for example an infusion rate which may exceed a maximum infusion rate as stored in the drug library, or which may lie below a minimum infusion rate as stored in the drug library. If this is found, the actual limits may be modified or proposed for modification in the drug library database to reflect the actually desired usage. The tendencies for all analysis changes, propositions or counter propositions may be stored in the knowledge database.

It also may be found that a particular drug is never used for a particular medical use, for example on cardiac patients. If this is found, for example a rule may be created and stored in the drug library database defining that the particular drug shall not be used for the specific medical use.

In instances where a certain medication is contra-indicated for certain types of conditions the knowledge database could be set up to automatically flag the medication and warn the user if the system knows of pre-existing condition that matches the criteria. Background of this is that drug libraries are separated into so-called care profiles. Each care profile corresponds to a service where general or specific patient conditions are treated. Drugs that are contra-indicated for a certain condition, but are part of a care profile can automatically be treated as hazardous. In addition, when precise information about a patient condition is available the prescription could be checked directly for contra indications (this could just be key word search in an internal or external database). The medication hence is flagged as potentially hazardous, but possibly an infusion using that medication is not blocked. The flagging can be done using the knowledge database.

By relating the actual usage date to particular environments, for example to a ward, a care area or an entire hospital, the drug library database may be modified differently for different environments to reflect the actual usage of a drug within the different environments. In this way, the drug library database is modified for example for different wards in a different fashion such that the system is adapted according to the actual use of medical devices in the particular environments.

The distribution server and the at least one medical device are, for example, placed in a common healthcare environment, for example within a hospital. The distribution server and the at least one medical device herein both are connected to a common communication network, for example a local area network (LAN) or a wireless local area network (WLAN) of the hospital. The server and the medical device hence communicate with each other via the communication network and exchange data via the communication network. In this way, drug library data may be transferred from the drug library database through the distribution server to the medical device via the communication network, and configuration and usage data entered into the medical device may be transferred from the medical device to the distribution server and the expert system comprising the knowledge database.

The object is also achieved by a method for operating a system for controlling the administration of a drug to a patient, the system comprising at least one medical device for administering a drug to a patient, a distribution server and a drug library database comprising drug library data. The method comprises:
programming the at least one medical device, according to drug library data of the drug library database, by entering configuration data for performing an administration operation for administering a drug to a patient.

Herein, the system records said configuration data and reports said configuration data to the distribution server, wherein the distribution server passes the configuration data to an expert system which stores said configuration data in a knowledge database and evaluates the knowledge database for modifying said drug library database according to said evaluation.

The evaluation of the knowledge database and all configuration data stored therein may, for example, take place by using a mathematical, for example statistical analysis. The analysis herein typically does not take place in real time, i.e., during a particular administration operation, but over an extended period of time in order to take configuration data received from multiple administration operations on multiple medical devices in a particular environment into account. According to the analysis, then, the drug library database may be modified in order to reflect the actual usage of medical devices in a healthcare environment. For this, a user may be prompted to consult the analysis results generated and accept the proposed modifications. Alternatively, the expert system may be set-up to perform the modifications on the drug library data directly. The expert system may log any modifications, including the time and date the modifications were applied, and may inform a user that a modification has been affected. A user may be enabled to create a rule set which may deviate from a proposal by the expert system by an acceptable margin.

The drug library database may for example store, for a multiplicity of drugs, boundary values defining limits for the programming of the at least one medical device. The boundary values may for example relate to a minimum or maximum infusion rate for administering a drug, a minimum or maximum time duration for administering a drug, or a minimum or maximum dosage for administering a drug. According to the boundary values a user may program a medical device to for example perform an infusion operation using a particular drug at a particular infusion rate for a particular time to administer a particular dosage. The infusion rate, the time and the dosage herein must lie within the boundary range defined for the particular drug in the drug library.

If, upon evaluating the configuration data stored in the knowledge database, it is found that the actual usage of the medical device differs from the settings and ranges stored in the drug library database, the drug library database may be modified accordingly. If it for example is found that typically a particular drug for a particular type of patient in a particular ward is used within a different range of infusion rates, the boundary values defined in the drug library database for this particular drug for the particular type of patient in the particular ward may be modified.

In addition, the drug library database may store information related to patient demographics, in particular a patient's age, gender and/or weight. In this way the expert system may be able to categorise medications in relation to specific patient demographics to determine the posology.

In addition, the drug library database may also store information related to a specific medical use of a drug. The medical use of a drug herein relates to the treatment of a specific disease or a specific condition of a patient. A drug may for example be used on cardiac patients or on patients having an influenza. Or a drug may be used for pain relieve or the like.

The drug library may for example store rules for administering a drug to a patient. The rules herein define a range of programming options for programming the at least one medical device for a specific drug and a specific patient. For example, a rule may be that a particular drug shall only be used for a specific type of patient, for example for cardiac patients. Another rule may be that a particular drug shall never be used in connection with another particular drug, due to the drugs not being compatible with each other.

Upon evaluating the configuration data stored in the knowledge database, rules may be modified or new rules may be created. If for example it is found that a particular drug is never used for a particular medical use, a rule may be created defining that the particular drug shall not be used for the particular medical use, for example for cardiac patients.

By means of the instant system and method an expert system is provided which may be operative to automatically modify drug library data stored in the drug library database. Hence, with the actual usage of medical devices within a healthcare environment, a drug library may continuously be improved according to actual usage data, wherein the evaluation and modification of the drug library data takes place using the knowledge database of the expert system, and upon modification new versions of drug libraries may be distributed via the distribution server to the different medical devices within a healthcare environment.

Some or all modifications to the drug library database, however, may be critical in that they may have severe consequences to the health of a patient. For example, if limits of a range of permissible infusion rates are increased, this must be done with care. Prior to entering any modification or at least a critical modification into a drug library database, hence, an information of and a confirmation by a user may be required, wherein for this the distribution server may communicate a message to a user requesting a confirmation for a particular modification of its drug library database.

The instant invention relates also to a software product implementing the method described above.

The idea underlying the invention shall subsequently be described in more detail with regard to the embodiments shown in the figure. Herein:

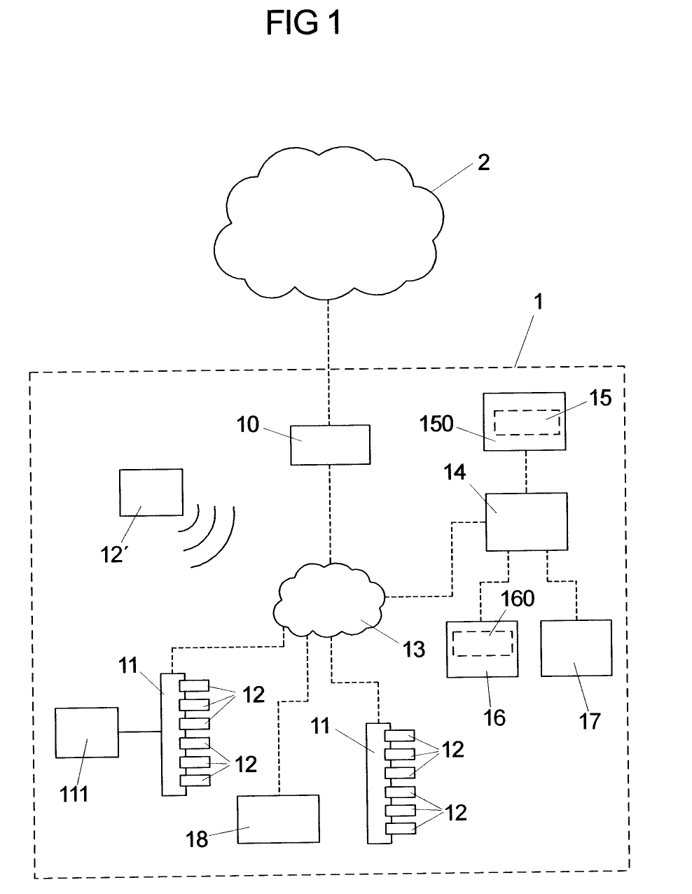

FIG. 1 shows a schematic view of medical devices located within a healthcare environment and communicatively connected to a distribution server;

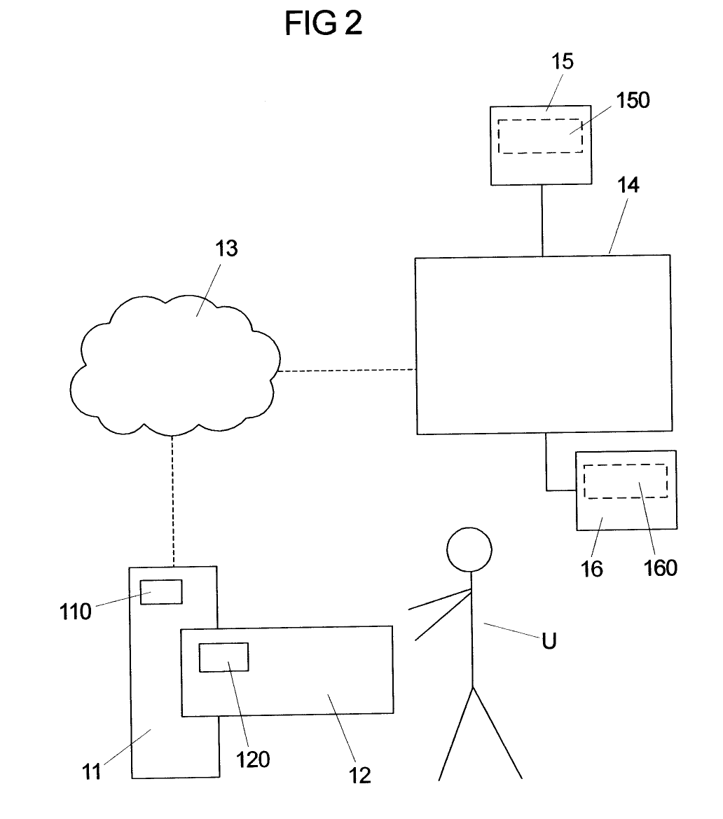

FIG. 2 shows a more detailed view of medical devices in connection with the distribution server;

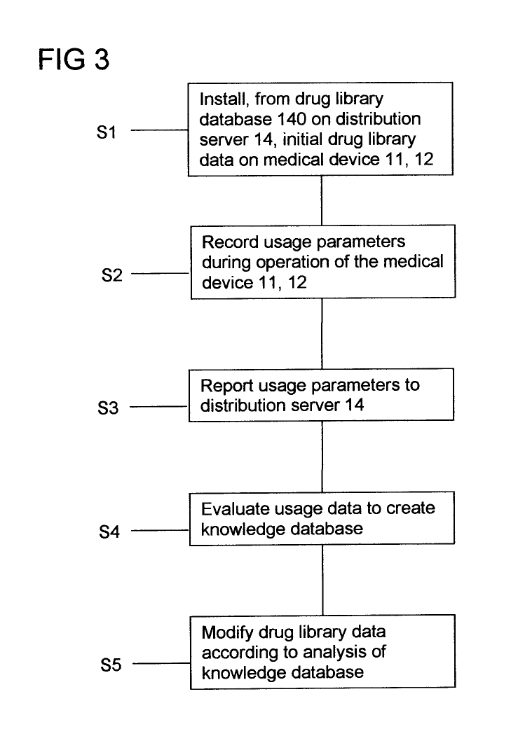

FIG. 3 shows a flow diagram of a method for operating the medical devices for performing an administration operation for administering a drug to a patient;

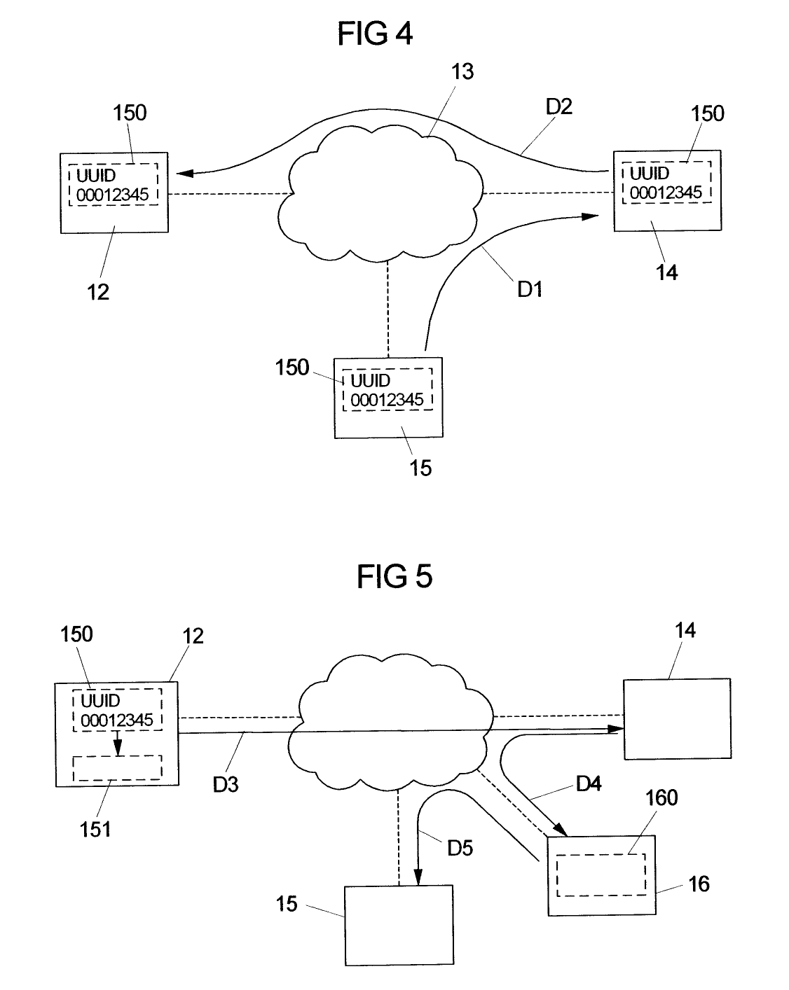

FIG. 4 shows a schematic view of a medical device interacting with a distribution server and a drug library database; and

FIG. 5 shows a schematic view of a medical device interacting with a distribution server, a drug library database and in addition an expert system.

FIG. 1 shows a schematic overview of a system for administering drugs to patients within a healthcare environment 1.

A healthcare environment 1 in this regard may be for example a hospital. The hospital may be organized to have different clinics, departments, wards and operation facilities and the like. Throughout the hospital a number of medical devices 11, 12, 12′ may be distributed serving to administer drugs to patients. Such medical devices 11, 12, 12′ may be infusion devices 12, 12′ such as infusion pumps in the shape of volumetric pumps or syringe pumps.

The infusion devices 12 may be connected to racks 11, wherein the racks 11 serve as mechanical carriers for the infusion devices 12 on the one hand and as communication links to facilitate communication between the infusion devices 12 and a hospital local network 13 on the other hand. Via the racks 11 and the local network 13, for example set up as a local area network (LAN), the infusion devices 12 may be connected to a hospital management system, hence allowing for a centralized management of the infusion devices 12 within the hospital local network 13.

To the rack 11 a control device called an infusion manager 111 may be attached. The infusion manager 111 may for example comprise an input device such as a touch-sensitive screen and may serve for example to enter control commands in relation to some or all of the infusion devices 12 attached to the rack 11.

Other infusion devices 12′ may be stand-alone devices which communicate with the hospital local network 13 for example wirelessly or wire-bound via suitable communication connections.

As shown in FIG. 2, the local network 13 may, via an interface 10, also be connected to an outside communication network 2, for example the internet. This is described for example in WO 2014/131729 A2, which shall be incorporated by reference herein.

A distribution server 14 is provided to transfer drug library data 150 to the medical devices 11, 12, 12′. Drug libraries, as they are conventionally known, serve to provide rules to medical devices 11, 12, 12′ for administering drugs to a patient. In particular, drug libraries in the context of the infusion devices 12, 12′ contain a list of drugs in which each drug is associated with parameters that define, characterize and impose boundary values on an infusion device 12, 12′ for administering the particular drugs to a patient. For example, such boundary values may relate to the dosage, the rate of administration and the time of administration for a drug and may vary for different drugs and also for different types of patients, for example dependent on the age, weight and gender of the patient.

The drug library data 150 are stored in a drug library database 15, which may be hosted on a separate database server or which may be installed on the distribution server 14. In the former case, the drug library database server and the distribution server 14 are separate physical entities. The distribution server 14 in this case serves as link to distribute the drug library data 150 from the drug library database 15 to the medical devices 11, 12, 12′. In the latter case, the drug library database 15 is installed as software on the distribution server 14 and is distributed from there to the medical devices 11, 12, 12′.

In one embodiment, drug libraries are installed on the medical devices 11, 12, 12′, in particular on infusion devices 12, 12′, such that during operation of an infusion device 12, 12′ parameters set by the drug library for a certain drug may be applied. For example, a nurse that wishes to program a particular infusion device 12, 12′ for performing an actual administration operation for infusing a drug to a patient may not be enabled to choose a dosage rate outside a range of dosage rates defined by the drug library for a certain drug. If a certain drug is to be administered to a patient, a nurse can chose administration parameters only within the boundaries defined by the drug library.

The drug library database 15 comprises one or multiple versions of drug libraries. Via the distribution server 14 drug libraries may be installed on the different medical devices 11, 12, 12′ in that the distribution server 14 transfers drug library data 150 via the local network 13 to the medical devices 11, 12, 12′ for installing the data on the medical devices 11, 12, 12′. The use of a distribution server 14 facilitates the distribution of drug libraries across the medical devices 11, 12, 12′ and also facilitates the updating of drug library data on the medical devices 11, 12, 12′, which may take place automatically by the distribution server 14 pushing updated data to the medical devices 11, 12, 12′ or by the medical devices 11, 12, 12′ pulling drug library data 150 via the distribution server 14.

In another embodiment, drug library data 151 may not be locally installed on a medical device 11, 12, 12′. Rather, the medical device 11, 12, 12′ may, during operation, query a drug library 150 on the drug library database 15. For example, if a user enters a medication into the medical device 11, 12, 12′ for programming the medical device 11, 12, 12′, the medical device 11, 12, 12′ may query the drug library 150 on the drug library database 150, and the programming of the medical device 11, 12, 12′ may take place using the drug library data 150 present in the drug library database 15. In this case, hence, no actual drug library is locally installed on the medical device 11, 12, 12′.

During actual use of a medical device 11, 12, 12′, a user U, for example a nurse or a physician, may for example enter configuration data into the medical device 11, 12, 12′ for programming the medical device 11, 12, 12′. By means of the configuration data parameters for performing an actual infusion operation are set, for example an infusion rate, a dosage and/or a duration for the infusion operation. In addition, the user U may identify herself, may be requested to identify the medical device 11, 12, 12′ and its location within the healthcare environment 1, may identify the patient, i.e. its name, gender, age and/or weight, and may identify a disease that shall be treated or the like.

A medical device 11, 12, 12′ may also be programmed using an external server 17 connected to the distribution server 14 directly are via the hospital network 13. The external server may for example be located in a pharmacy of the hospital environment 1 or may constitute a so-called auto-programming system. For programming the medical device 11, 12, 12′, a user may for example enter configuration data into the server 17, upon which the configuration data is routed via the distribution server 14 to the respective medical device 11, 12, 12′. The programming of the medical device 11, 12, 12′ hence does not take place locally at the medical device 11, 12, 12′, but remotely via the server 17.

Within the instant system, all configuration data that is used for operating the medical device 11, 12, 12′ is recorded for example via a control device 120 of the infusion device 11, 12, 12′ or a control device 110 of the rack 11 carrying the infusion device 11, 12, 12′, and the configuration data (a record of input data or operating data) is transferred via the local network 13 to the distribution server 14. The configuration data is entered into a knowledge database 160 of an expert system 16, which stores configuration data of a multiplicity of infusion operations on a multiplicity of medical devices 11, 12, 12′ and hence contains a record of actual usage data over an extended period of time within the healthcare environment 1.

The configuration data may be locally recorded via a suitable control device 110, 120 at the respective medical device 11, 12, 12′. If the configuration data is routed to the medical device 11, 12, 12′, however, the configuration data may also be recorded as it passes through the distribution server 14. Hence, the configuration data is not locally recorded at the medical device 11, 12, 12′, but is recorded on its way passing through the distribution server 14 as transient data.

By means of the expert system 16 comprising the knowledge database 160 a self-learning system may be provided. In particular, the configuration data stored in the knowledge database 160 may be evaluated by the expert system 16, namely a control software arranged thereon, such that the drug library database 15 may be modified according to the results of the evaluation.

The expert system 16 may reside on a separate server. However, it instead is also possible that the expert system 16 with its knowledge database 160 is hosted on the distribution server 14 and hence is part of the distribution server 14. In that case, the expert system 16 is installed as software on the distribution server 14.

A flow diagram of a method implementing such a modification procedure of the drug library database 15 is shown in FIG. 3.

Initially (step S1), drug library data 150 may be installed on one or multiple medical devices 11, 12, 12′ from the drug library database 15 installed on the distribution server 14. Drug library data herein may be distributed from the distribution server 14 to all medical devices 11, 12, 12′ within a healthcare environment 1, wherein in different areas of the healthcare environment 1, for example in different clinics, wards or care areas, different drug libraries may be installed on the respective medical devices 11, 12, 12′.

This is further illustrated in FIG. 4. Within the drug library database 15 an actual version of a drug library 150 may be present having a unique identifier (UUID) 00012345. If the drug library 150 has been updated, the drug library database 15 may push the drug library 150 via the distribution server 14 towards one or multiple medical devices 12. Alternatively, a medical device 12 may query the drug library database 15 whether a unique identifier of a drug library 150 installed on the medical device 12 matches the unique identifier of the latest version of the drug library 150 in the drug library database 15. If there is a mismatch between the unique identifier of the drug library 150 on the medical device 12 and the unique identifier of the latest version of the drug library 150 in the drug library database 15, the medical device 12 may request the latest version of the drug library 150 to be downloaded to the medical device 12 via the distribution server 14. For this, the drug library 150 is transferred to the distribution server 14 (data stream D1) and from the distribution server 14 on to the medical device 12 (data stream D2).

According to the drug library data 150 installed on a medical device or contained in the drug library database 15, a user U may then program the medical device 11, 12, 12′ for performing an actual infusion operation. For this the user U enters configuration data into the medical device 11, 12, 12′, or configuration data may be routed to the medical device 11, 12, 12′ from the external server 17, wherein the configuration data may for example comprise an actual infusion rate, a dosage, information relating to the patient and to the user or the like. For this, the user U initially selects a drug, wherein upon the selection of the drug boundary values are loaded from the drug library data 150 installed on the medical device 11, 12, 12′ or queried from the drug library database 15 such that the user U may for example set the infusion rate only within the limits provided by the boundary values stored in the drug library data 150.

All configuration data 151 that is entered into the medical device 11, 12, 12′ is recorded by the control device 120 at the infusion device 12 or by the control device 110 at the rack 11, or—if the configuration data 151 is routed to the medical device 11, 12, 12′ from an external server 17 via the distribution server 14—is recorded as it passes through the distribution server 14 as transient data (step S2). The configuration data 151 is then passed via the distribution server 14 to the expert system 16 which enters the configuration data 151 into the knowledge database 160 (step S3).

This is illustrated in FIG. 5. If configuration data 151, upon actual usage of the medical device 12, is present at the medical device 12, this configuration data 151 is transferred from the medical device 12 towards the distribution server 14 (data stream D3). The distribution server 14 passes the configuration data 151 on to the expert system 16 (data stream D4), which stores the configuration data 151 in its knowledge database 160.

This is repeated for all infusion operations that take place within a healthcare environment 1 over an extended period of time such that the knowledge database 160 is continuously filled with configuration data 151 according the actual usage of medical devices 11, 12, 12′ within the healthcare environment 1.

Using the information stored in the knowledge database 160, a mathematical analysis may be carried out, and according to the analysis the data stored in the drug library database 15 may be modified to reflect the actual usage of the medical devices 11, 12, 12′ in the healthcare environment 1 (steps 4 and 5 in FIG. 3). Hence, as shown in FIG. 5, drug library data 150 installed on the drug library database 15 may automatically and without further user interaction be modified by means of the expert system 16 (data stream D5). Alternatively, the drug library database 15 may be informed that a modification of drug library data 150 is proposed by the expert system 16, which however is entered into the drug library database 15 only upon confirmation by one or multiple users.

For example, boundary values may be altered, new rules may be defined or existing rules may be modified.

If for example it is found that a particular drug is never used on cardiac patients, a rule may be defined stating that the particular drug shall not be used on cardiac patients.

If for example it is found that users repeatedly request for example a dosage for a particular drug for a particular type of patients, for example cardiac patients, that lies outside a range defined in the drug library database 15, the range may be modified to allow the selection of such dosage.

If it is found that typically a selected infusion rate for a particular drug for a particular type of patient lies within a much smaller range than defined by the drug library, the range in the drug library database 15 may be modified accordingly.

The modification of the drug library database 15 may take place taking the actual usage data of different clinics, wards or care areas into account. For example, in an intensive care unit the usage data may significantly differ from the usage data in an oncology care area. Hence, the drug library database 15 may be modified differently for the intensive care unit care area and the oncology care area.

The idea underlying the invention is not limited to the embodiments described above.

In particular, medical infusion devices not necessarily are placed on racks, but may be used also in a stand-alone fashion in a care area.

The distribution server typically is a computer system placed in a healthcare environment. The method described herein may be implemented by a software installed on the distribution server, the software being adapted to carry out an analysis of the knowledge database for modifying the drug library database.

### LIST OF REFERENCE NUMERALS

1 Healthcare environment
10 Interface
11 Medical device (rack)
110 Control device
111 Infusion manager
12, 12′ Medical device (infusion pump)
120 Control device
13 Local network
14 Distribution server
15 Drug library database
150 Drug library data
151 Configuration data
16 Expert system
160 Knowledge database
17 Third party system
18 Client machine
2 Public communication network
D1-D5 Data transfer
S1-S5 Steps
U User

1. A system for controlling the administration of a drug to a patient, the system comprising:
at least one medical device for administering a drug to a patient,
a distribution server, and
a drug library database comprising drug library data, the at least one medical device being programmable, according to the drug library data of the drug library database, by entering configuration data into the at least one medical device for performing an administration operation for administering the drug to the patient,
wherein the system is operative to record said configuration data and to report said configuration data to the distribution server, wherein the distribution server is configured to pass said configuration data to an expert system comprising a knowledge database for storing said configuration data, the expert system being configured to evaluate the knowledge database for modifying said drug library database according to said evaluation.

2. The system according to claim 1, wherein the at least one medical device comprises an infusion device for infusing a medical liquid to a patient.

3. The system according to claim 1, wherein the at least one medical device comprises a rack on which a multiplicity of infusion devices are arrangeable.

4. The system according to claim 1, wherein the distribution server and the at least one medical device are communicating via a communication network.

5. A method for operating a system for controlling the administration of a drug to a patient, the system comprising at least one medical device for administering a drug to a patient, a distribution server and a drug library database comprising drug library data, the method comprising:
programming the at least one medical device, according to the drug library data of the drug library database, by entering configuration data into the at least one medical device for performing an administration operation for administering the drug to the patient, and
recording said configuration data and reporting said configuration data to the distribution server, wherein the distribution server passes the configuration data to an expert system which stores said configuration data in a knowledge database and evaluates the knowledge database for modifying said drug library database according to said evaluation.

6. The method according to claim 5, wherein the drug library database stores, for a multiplicity of drugs, boundary values defining limits for the programming of the at least one medical device.

7. The method according to claim 6, characterized in that wherein the boundary values relate to at least one of
a minimum and/or maximum infusion rate for administering a drug,
a minimum and/or maximum time duration for administering a drug, and
a minimum and/or maximum dosage for administering a drug.

8. The method according to claim 6, wherein the expert system, upon evaluating said configuration data stored in the knowledge database, is operative to modify the stored boundary values.

9. The method according to claim 5, wherein the drug library database stores patient demographic information related to at least one of the patient's age, gender and weight.

10. The method according to claim 5, wherein the drug library database stores information related to a specific medical use of a drug.

11. The method according to claim 5, wherein the drug library database stores rules for administering a drug to a patient, the rules defining a range of programming options for programming the at least one medical device for a specific drug and a specific patient.

12. The method according to claim 11, wherein the expert system, upon evaluating said configuration data stored in the knowledge database, is operative to modify the rules stored in the drug library database.

13. The method according to claim 5, wherein the expert system enters a modification into the drug library database automatically or upon confirmation by a user.

14. A non-transitory carrier medium carrying computer executable code that, when executed on a server, causes said server to perform the method according to claim 5.
